(19) United States
(12) Patent Application Publication
(10) Pub. No.: US 2026/0232170 A1
(43) Pub. Date: Aug. 13, 2026

(54) PROVISIONING SYSTEM FOR ENDOSCOPES

(71) Applicant: OLYMPUS Winter & Ibe GmbH, Hamburg (DE)

(72) Inventors: Sascha JASKOLA, Hamburg (DE); Ralf SIEGMUND, Ahrensburg (DE); Daniel ZUEWERS, Hamburg (DE); Jan NIEBUHR, Kiel (DE); Christoph Alexander AHRENS, Reinbek (DE); Mathias HUEBER, Hamburg (DE); Ralf TESSMANN, Hamburg (DE); Stefan SCHROEDER, Hamburg (DE); Veronika STEFKA, Hamburg (DE); Jaron SINGHAL, Hamburg (DE)

(73) Assignee: OLYMPUS Winter & Ibe GmbH, Hamburg (DE)

(21) Appl. No.: 19/636,247

(22) Filed: Apr. 1, 2026

Related U.S. Application Data
(63) Continuation-in-part of application No. 17/963,433, filed on Oct. 11, 2022, now abandoned.
(60) Provisional application No. 63/255,047, filed on Oct. 13, 2021.

(51) Int. Cl. A61B 1/00; A61B 1/015

(52) U.S. Cl. CPC A61B 1/00006; A61B 1/0002; A61B 1/015

(57) ABSTRACT

Systems and methods for operating an endoscope reprocessing device are described. A processor can receive telemetry from sensors of the endoscope reprocessing device indicating physical process parameters currently being used by the endoscope reprocessing device. The processor can execute machine learning models, with inputs based on the telemetry, to determine an optimization target indicating target physical process parameters for optimizing operations of the endoscope reprocessing device. The processor can generate modified physical process parameters based on the physical process parameters indicated by the telemetry and the optimization target. The processor can convert the modified physical process parameters into actuator level control commands that control actuators of the endoscope reprocessing device. The processor can encode the actuator level control commands in digital signals. The processor can transmit the digital signals to the endoscope reprocessing device to cause the actuators to perform a reprocessing step according to the modified physical parameters.

### CROSS-REFERENCE TO RELATED APPLICATIONS

The present application is a continuation-in-part of U.S. patent application Ser. No. 17/963,433 filed on Oct. 11, 2022, which claims the benefit of U.S. Provisional Patent Application No. 63/255,047 filed on Oct. 13, 2021, the entire contents of each of which is incorporated herein by reference.

### BACKGROUND

### Field

The present disclosure relates to a provisioning system for endoscopes.

### Prior Art

Endoscopes have long been used in medicine to examine or treat cavities of a patient's body that are difficult to access. They are generally reusable and must undergo a complex reprocessing process after each use before they can be used to examine or treat another patient.

A reprocessing process, e.g., for gastroenterological endoscopes, usually includes manual pre-cleaning, machine cleaning and disinfection, drying if necessary, and storage in a supply cabinet.

Offices or departments specialized in endoscopic procedures regularly comprise a large number of treatment rooms in which several procedures can be performed in parallel. For this purpose, a large number of endoscopes of different types are kept in stock, and several manual and mechanical reprocessing stations are provided for their reprocessing. Within the framework of occupancy planning, it is determined for the individual treatment rooms when which procedures are to be carried out with which endoscopes or endoscope types, and the patients to be treated in each case are appointed accordingly. In the occupancy planning, it can also be determined at which reprocessing stations the endoscopes are reprocessed after use.

In occupancy planning, some parameters must first be estimated. These include, for example, the duration of individual procedures and the duration of reprocessing of individual endoscopes before they can be used again. Deviations in the actual duration of a procedure or a reprocessing process can have significant consequences if, for example, an endoscope is not available in time to be subjected to machine cleaning and disinfection together with other endoscopes after manual pre-cleaning due to a longer procedure. In this case, either the start of the machine cleaning and disinfection must be delayed, as a result of which all the endoscopes concerned are not available for reuse until later, or the endoscope that is not available until later must be provided for a later run of the machine cleaning and disinfection, as a result of which the re-provision of the endoscope concerned is delayed even further.

A system for managing endoscopes is known from U.S. Pat. No. 8,768,721 B2, which determines the possible effects of a reprocessing capacity failure on occupancy planning and uses this to determine an additional requirement for endoscopes, which are then procured as items on loan or on purchase. However, it is not always possible to procure additional endoscopes at short notice.

However, it is difficult for a person responsible for occupancy planning to foresee the actual impact of delays in individual procedures or reprocessing processes on current occupancy planning.

Furthermore, the estimated values of the individual process durations available for occupancy planning allow only limited accuracy in planning.

### SUMMARY

Therefore, an object is to develop an improved provisioning system for endoscopes.

Such objective can be achieved by a provisioning system for endoscopes in an application environment comprising one or more examination rooms and one or more reprocessing stations for endoscopes, the provisioning system being configured to receive first information about scheduled procedures and to receive second information about a status of one or more endoscopes, the second information comprising an indication of whether an endoscope is ready for use, currently in use, or currently being reprocessed. The provisioning system further can comprise a user interface having a graphical user interface, wherein the graphical user interface can include a first presentation area in which data about ready-to-use endoscopes is displayed, a second presentation area in which data about scheduled procedures is displayed, and a third presentation area in which data about endoscopes undergoing reprocessing is displayed.

Receiving the second information may comprise receiving data that does not directly describe the state of an endoscope but allows conclusions to be drawn about it, and further evaluating this data to determine the state of the endoscope. Additionally, a location of an endoscope may also be determined.

By the appropriate embodiment of the provisioning system, information about the availability of the endoscopes can be offered in such a way that a user can intuitively grasp it and immediately recognize whether a required endoscope will be available in time for all planned procedures.

In this context, the first presentation area, the second presentation area, and the third presentation area may be arranged along a main direction, wherein the third presentation area is arranged between the first presentation area and the second presentation area.

Data for individual endoscopes and/or procedures in the presentation areas may be arranged along a second main direction which is perpendicular to the first main direction. A corresponding grid-like arrangement of the information can make it easy for a user to grasp.

In an embodiment of a provisioning system, the data in the first presentation area, the second presentation area, and the third presentation area may be arranged such that data relating to an endoscope presented in the first presentation area or the third presentation area is aligned along the second main direction with data relating to a procedure in the second presentation area in which the respective endoscope is to be used. Acquisition of interrelated information is thereby further simplified.

In another embodiment of a provisioning system, a visualization may be displayed in the third presentation area for each endoscope undergoing a reprocessing procedure reflecting the progress of the reprocessing procedure.

The visualization for each of a plurality of steps of a reprocessing process may comprise a visualization element indicating whether the respective step is completed, in progress, or pending.

In the third presentation area, for each endoscope in a reprocessing process, an expected time at which the endoscope will be available may be displayed.

In a further embodiment of a provisioning system, the provisioning system may be configured to take into account historical data of previous reprocessing processes when determining a time at which an endoscope is expected to be available. In this way, the time can be determined reliably.

The provision system may be configured to store and/or statistically evaluate the duration of reprocessing processes that have been performed.

Such object can also be achieved by a method for operating a provisioning system for endoscopes, comprising: receiving first information about scheduled procedures, receiving second information about a status of one or more endoscopes, the second information comprising an indication of whether an endoscope is ready for use, currently in use, or currently being reprocessed, and displaying the first and second information in a graphical user interface. the graphical user interface can include a first presentation area in which data about ready-to-use endoscopes is displayed, a second presentation area in which data about scheduled procedures is displayed, and a third presentation area in which data about endoscopes undergoing reprocessing is displayed.

Other objectives can be achieved by a provisioning system for endoscopes in an application environment comprising one or more examination rooms and one or more reprocessing stations for endoscopes, the provisioning system being configured to receive first information about scheduled procedures and to receive second information about a status of one or more endoscopes, the second information comprising an indication of whether an endoscope is ready for use, currently in use, or currently being reprocessed, and wherein the provisioning system is configured to determine from the first information and the second information whether a ready-to-use endoscope is or will be available for each of the scheduled procedures. The provisioning system can further comprise a user interface having a graphical user interface, wherein the graphical user interface can include a first presentation area in which data about ready-to-use endoscopes is displayed, a second presentation area in which data about scheduled procedures is displayed, and a third presentation area in which data about endoscopes undergoing reprocessing is displayed, wherein the data displayed in the second presentation area comprises an indicator which indicates whether a ready-to-use endoscope is or will be available for the respective procedure.

Receiving the second information may comprise receiving data that does not directly describe the state of an endoscope but allows conclusions to be drawn about it, and further evaluating this data to determine the state of the endoscope. Additionally, a location of an endoscope may also be determined.

By the appropriate embodiment of the provisioning system, information about the availability of the endoscopes can be offered in such a way that a user can intuitively grasp it and immediately recognize whether a required endoscope will be available in time for all planned procedures.

In a further embodiment of a provisioning system, the provisioning system may be configured, in a case in which an endoscope will not be available in time for a scheduled procedure, to determine possible modifications to ongoing or pending reprocessing processes by which a delay in the provision of the endoscope concerned can be reduced.

The provisioning system may be configured to determine the possible modifications upon request by a user. Further, the provisioning system may be configured to provide determined possible modifications in the form of a selection list.

In this way, in addition to the information as to whether an endoscope will be available for all procedures, a user of the provisioning system can be quickly and easily offered possible solutions in the event of problems with the provision, in order to reduce or even completely avoid influences of the delayed provision on the examination procedure.

The provisioning system may be configured to generate and send control commands to an endoscope reprocessing device upon selection of a determined modification by the user in order to cause the endoscope reprocessing device to perform a modified reprocessing process. Similarly, the provisioning system may be configured, upon selection of a determined modification by the user, to generate and transmit execution instructions for a modified reprocessing process in text form to a manual pre-cleaning station for display.

Depending on whether the preparation process or preparation step to be modified is a process or process step performed by machine or manually, the modification can thus be implemented easily and effectively.

The provision system may comprise a control memory in which rules are stored according to which process parameters of reprocessing processes can be modified without impairing the effectiveness of the reprocessing processes.

Performance curve fields may be stored in the control memory, which describe the effectiveness of a reprocessing step as a function of one or more process parameters.

By a corresponding control memory and, if applicable, performance curve fields stored therein, possible modifications of reprocessing processes can be determined effectively.

According to another aspect, such object can be achieved by a method for operating a provisioning system for endoscopes, comprising: receiving first information about scheduled procedures, receiving second information about a status of one or more endoscopes, the second information comprising an indication of whether an endoscope is ready for use, currently in use, or currently being reprocessed, and determining, from the first information and the second information, whether a ready-to-use endoscope is or will be available for each of the scheduled procedures, and displaying the first and second information in a graphical user interface. Therein, the graphical user interface can include a first presentation area in which data about ready-to-use endoscopes is displayed, a second presentation area in which data about scheduled procedures is displayed, and a third presentation area in which data about endoscopes undergoing reprocessing is displayed, wherein an indicator can displayed in the second presentation area, which indicates whether a ready-to-use endoscope is or will be available for the respective procedure.

With regard to possible further developments as well as the advantages and effects achievable thereby, express reference is made to what has been said above.

### BRIEF DESCRIPTION OF THE DRAWINGS

In the following, embodiments of a reprocessing system for endoscopes will be described in more detail with reference to some exemplary figures. In this regard, the illustration serves only to provide a better understanding of the invention without limiting it, in which:

FIG. 1 illustrates a provisioning system for endoscopes,

FIG. 2 illustrates a user interface,

FIG. 3 illustrates another user interface,

FIG. 4 illustrates a performance curve field,

FIG. 5 illustrates details of the provisioning system shown in FIG. 1 in one embodiment, and

FIG. 6 illustrates a process for adaptively operating an endoscope reprocessing device in one embodiment.

### DETAILED DESCRIPTION

The systems and methods described in the present disclosure provide machine-to-machine control and physical transformation where artificial intelligence (AI) modules can produce time-stamped ready predictions, generate concrete control commands or execution instructions to reprocessing devices/manual stations, and modify physical process parameters based on stored performance curves.

Further, the AI modules can improve conventional reprocessing systems, such as improving prediction accuracy for endoscope readiness by using additional information and still-image progress estimation, reducing false sensor-state interpretations via sensor fusion, and prevents physical failures through anomaly detection.

The still-image progress estimation utilizes a time in which the last image is taken during the operation/procedure or during the withdrawal of the endoscope in order to determine a start time of a pre-cleaning process.

Furthermore, graphical user interface (GUI) and rules specific to the AI-based system described herein provides AI results in specifically defined areas in the GUI to allow users to perform modifications based on decision artifacts stored in control memory, allowing users to select appropriate AI actions (recorded machine actions or machine-prepared options that can be executed).

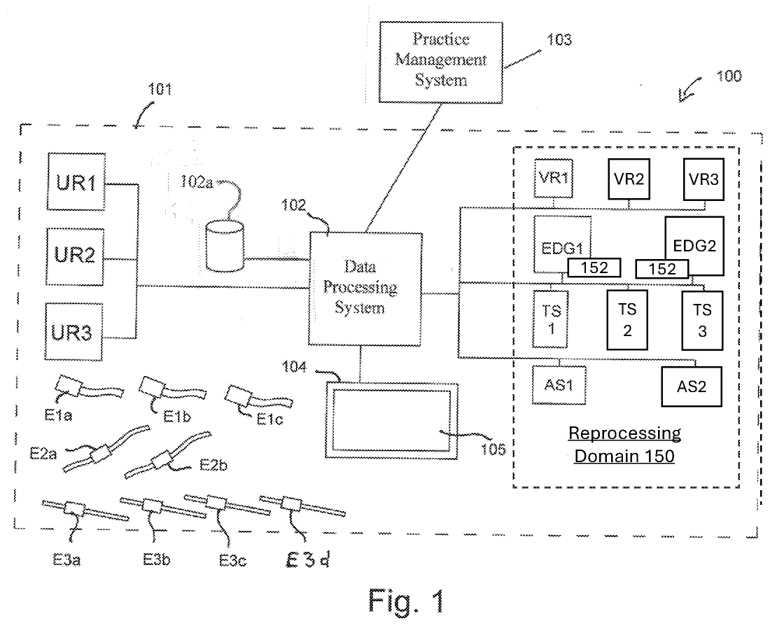

FIG. 1 shows an endoscope provisioning system 100 in an exemplary application environment 101, which may be an outpatient endoscopy office or an endoscopy department of a hospital.

The application environment includes a plurality of examination rooms UR1, UR2, UR3. The number of examination rooms may vary as desired. The term “examination room” does not exclude that procedures with interventional parts such as biopsies or sclerotherapy are also performed in these rooms.

Furthermore, the application environment comprises a reprocessing station for the reprocessing of used endoscopes, which includes manual pre-cleaning stations VR1, VR2, VR3, endoscope reprocessing units EDG1, EDG2, drying cabinets TS1, TS2, TS3, and storage cabinets AS1, AS2. The numbers of the individual elements are again arbitrary and serve only as an example. Endoscope reprocessing devices EDG1, EDG2, can be Automated Endoscope Reprocessors (AERs), configured to clean and disinfect endoscopes using high-level disinfectants (HLD) or liquid chemical sterilants. Endoscope reprocessing devices EDG1, EDG2, can also be configured to perform automated leak testing (e.g., detect damage before the disinfecting process) and channel monitoring (e.g., automatically detect channel blockages and connection faults to ensure comprehensive cleaning). Each one of EDG1, EDG2 can include a respective reprocessing processor 152 (“processor 152”). Processor 152 can be embodied by software running on a processor, controller, CPU or regular or special purpose computer (e.g., medical-grade computer) and each may have an associated storage device (memory). Each one of endoscope reprocessing devices EDG1, EDG2, can include a basin for housing an endoscope to be cleansed and disinfected. Processor 152 in each one of endoscope reprocessing devices EDG1, EDG2, can be configured to control various aspects of the endoscope reprocessing, such as monitoring and setting temperature and pressure set points, durations of sub-processes, being used in the endoscope reprocessing, image processing within the basin or outside such as in the drying cabinets and storage cabinets, etc. In an aspect, an endoscope reprocessing can include at least one sub-process, such as manual cleaning, leakage test, disinfection, sterilizing, cleaning, rinsing, drying, storage, etc.

A practical controller embodiment is a finite state machine (FSM) that formalizes stages of a reprocessing cycle (for example: PRE-CLEAN→WASH→RINSE→DRY→READY). Each state has minimum and maximum dwell times and guarded transitions that may be triggered either by scheduled workflow timing or by sensor/fusion/optimizer outputs. Example safety rules that the FSM enforces include: (a) never command a heater setpoint above a material safety temperature Tmax; (b) on any communication failure or missing device acknowledgment the FSM transitions to a SAFE-HOLD state and requires manual operator verification; and (c) if a sensor anomaly exceeds a configured anomaly threshold for a defined period the FSM issues a pause/hold command and logs the event. The FSM design supports deterministic device behavior and clear, testable safety envelopes for automated modifications to reprocessing steps.

To perform endoscopic examinations in the examination rooms UR1, UR2, UR3, several endoscopes of different types are kept available. For example, endoscopes E1a, E1b, E1c of a first type E1, endoscopes E2a, E2b of a second type E2, and endoscopes E3a, E3b, E3c, E3d of a third type E3 are kept available. The endoscope types E1, E2, and E3 can be gastroscopes, bronchoscopes, and colonoscopes. Of course, endoscopes of other types may also be present.

The provisioning system 100 includes a data processing system 102 configured to receive and process information related to scheduled and/or ongoing procedures and reprocessing processes, as well as the current location and condition of individual endoscopes, if applicable. The data processing system 102 can be embodied by software running on a processor, controller, CPU or regular or special purpose computer (e.g., medical-grade computer) and each may have an associated storage device (memory). Data processing system 102 can communicate with processor 152 of EDG1, EDG2, to facilitate operations of provisioning system 100. In one embodiment, processor 152 can be configured to control the endoscope reprocessing operations of EDG1, EDG2 based on various digital signals provided by data processing system 102 (described below).

Information about scheduled procedures may be provided to the data processing system 102, for example, by a hospital or office management system 103. The office management system 103 can be embodied by software running on a processor, controller, CPU or regular or special purpose computer (e.g., medical-grade computer) and each may have an associated storage device (memory). The information may be provided via an interface, for example using the Digital Imaging and Communications in Medicine (DICOM) standard. Such information may include the type of procedure, the scheduled start of the procedure, and the examination room in which the procedure is to be performed. In one embodiment, office management system 103 can send a DICOM file including digital data that is a compilation of image pixel data with comprehensive, standardized metadata such as patient info, study details, and technical parameters. The DICOM file can also include digital data defining protocols for querying, retrieving, storing, and printing images over TCP/IP or HTTP(S) networks. The DICOM file can include digital data that is not readable by humans, but can be readable by processor 152. Processor 152 can decode the DICOM file and read the decoded data to control reprocessing in reprocessing domain 150.

Information about ongoing reprocessing procedures is usually provided by the endoscope reprocessing equipment.

To determine the location of individual endoscopes, sensors are provided in the examination rooms UR1, UR2, UR3, as well as at the reprocessing stations, which detect identification features of endoscopes located in the examination room or at the reprocessing station. Such sensors may include, for example, radio frequency identification (RFID) readers that detect RFID tags individually assigned to the endoscopes, either automatically or manually controlled.

The provisioning system may incorporate a sensor-fusion engine that combines signals from multiple sensors, including humidity sensors, optical wetness detectors, pressure sensors, temperature probes, and RFID-based location sensors. Fusion rules may assign weights to each data source or use probabilistic inference methods to derive a unified state classification. For example, if a humidity sensor reports dryness but an optical detector identifies residual water droplets, the fusion engine may classify the endoscope as “wet” with a weighted confidence, prompting the provisioning system to generate an “EXTEND_DRYING” command.

The system may also detect anomalies such as pressure spikes in the disinfectant line, abnormal heating profiles, or inconsistent sensor readings that deviate from learned patterns. Upon anomaly detection, the processor may issue a “PAUSE_CYCLE” or “ENTER_SAFE_STATE” command instructing the reprocessing device to halt the ongoing step, isolate the endoscope, and await further evaluation. These fusion-derived decisions and anomaly-triggered actions are stored in the control memory, ensuring traceability, auditability, and improved operational safety.

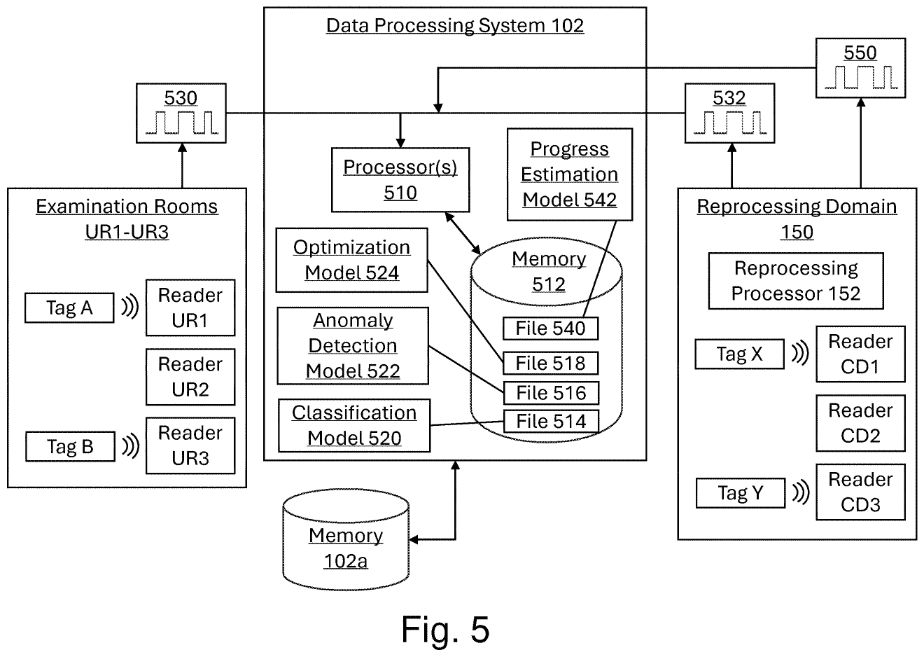

In one embodiment shown in FIG. 5, examination rooms UR1, UR2, UR3 can be installed with RFID readers labeled as Reader UR1, Reader UR2, Reader UR3, respectively. Further, in a reprocessing domain 150, which is a physical space, each one of manual pre-cleaning stations VR1, VR2, VR3, endoscope reprocessing units EDG1, EDG2, drying cabinets TS1, TS2, TS3, and storage cabinets AS1, AS2, can also be installed with RFID readers, such as Reader CD1, Reader CD2, Reader CD3 shown in FIG. 5. Data being read from the RFID readers in reprocessing domain 150 can be directly provided to data processing system 102. Data processing system 102 can analyze and process the data from the RFID readers to generate commands for controlling components in reprocessing domain 150.

Each one of the RFID readers, as shown in FIG. 5, can be configured to emit electromagnetic energy to power RFID tags on the endoscopes located in a corresponding location. Each RFID tag on the endoscopes can, in response to receiving electromagnetic energy from a specific RFID reader, return data to the specific RFID reader as radio frequency (RF) signals. Each one of the RFID readers shown in FIG. 5 can include, for example, a signal generator configured to generate a RF signal and periodically broadcast the RF signal in a corresponding physical space. Further, each one of the RFID readers shown in FIG. 5 can include a receiver configured to detect and receive RF signals wirelessly from RFID tags on the endoscopes. Also, each one of the RFID readers shown in FIG. 5 can include a controller, such as a microcontroller, configured to filter the received RF signals to remove noise and to convert the filtered signals from analog domain into digital domain, generating digital signals that can be transmitted to data processing system 102 as digital data that can be interpreted and decoded by hardware processors in data processing system 102.

In the embodiment shown in FIG. 5, the RFID readers in the examination rooms can output at least one digital signal 530 encoding, for example, IDs of the endoscopes and RFID reader location and/or ID. The RFID readers in the reprocessing domain 150 can output at least one digital signal 532 encoding, for example, IDs of the endoscopes and RFID reader location and/or ID. Digital signals 530, 532 can be transmitted and stored as binary bits encoding hexadecimal representation of the endoscope and location IDs. Data processing system 102 can include various network components and receivers that can receive the digital signals 530, 532 under various protocols. For example, the RFID readers can transmit digital signals 530, 532 to data processing system 102 by, for example, transmitting via standard wired or wireless communication interfaces such as USB, RS232/485, Ethernet, Wi-Fi, or Bluetooth.

Focusing on data processing system 102 in FIG. 5, data processing system 102 can include one or more processors including a processor 510 and at least one memory device including a memory 512. The one or more processors, including processor 510, can include CPUs, GPUs, TPUs, various types of AI accelerators, and/or other types of devices and processing units including hardware configured to perform arithmetic, logic, control, and input/output (I/O) operations of the computing device housing data processing system 102. Memory 512 can include various types of storage devices, such as volatile memory devices, non-volatile memory devices, caches, registers, Random Access Memory (RAM), virtual memory, or other types of memory devices for implementing the systems and methods described herein. Memory 512 can be configured to store program code, such as source code and/or execution code, that can be read and executed by processor 510 to perform the methods and various operations described herein.

Memory 512 can be configured to store a file 514 that is a digital representation of a classification model 520. In one embodiment, classification model 520 can be a probabilistic classifier, such as a Bayesian classifier, that can be trained using known or labeled historical sequences and events in the examination rooms and the reprocessing domain 150. File 514 can be, for example, a binary serialized file, such as Hierarchical Data Format version 5 (HDF5) or JavaScript Object Notation (JSON), that includes a structured collection of data such as arrays or tensors of floating-point numbers representing model weights, parameters, and hyperparameters. File 514 can also include program code, that are machine readable codes not interpretable by humans, that define the decision boundaries or probabilities used to categorize input data being inputted to classification model 520 into predefined classes.

Processor 510 can run classification model 520, either for training classification model 520 or for using classification model 520 to perform classification, by executing the program code in file 514. Execution of the program code in file 514 can allow processor 510 to perform computations on a set of inputs and the execution can result in classification model 520 outputting a classified result. In the training phase, the inputs can be training data that are labeled or unlabeled (e.g., supervised or unsupervised training) and the outputs can be fed back to the classification model 520 to be compared with an expected output for determining an error. This training process can repeat until the error converges to a target error amount or complies with a goal or objective. Once classification model 520 is trained and deployed, processor 510 can run classification model 520 for classification, such as inputting real world input data and running the trained classification model 520 to classify the input. Processor 510 can be configured to train various other models stored in memory 512 in a similar manner.

Processor 510 can decode digital signals 530, 532 to extract the endoscope and location IDs. Processor 510 can run classification model 520 using the extracted information as input to perform classification of the input. By using classification model 520 to classify input information indicative of ID of endoscopes and ID of RFID readers installed in different locations, false positives or negative results from intermittent sensor reads can be reduced, and explicit, auditable state labels that downstream modules (e.g., scheduling, automated reprocessing commands) can be provided for making deterministic decisions. The system already stores scope location/status and present reprocessing states in the third presentation area; the classifier concretely processes those signals to produce machine-actionable state labels.

Information about the current status of individual endoscopes is largely derived from the above. For example, if an endoscope is in an examination room UR1, the digital signal 530 being provided by the RFID readers in the examination room UR1 will encode an ID of the RFID tag on the endoscope and an ID of the RFID reader in examination room UR1. Processor 510 can generate a timestamp indicating a receiving time of digital signal 530, and decode digital signal 530 to extract the encoded information. Processor 510 can run classification model 520 using the extracted information as input to determine a status that the endoscope is in-use.

If, on the other hand, the endoscope is at a manual pre-cleaning station, the state may be assumed to be that the endoscope is currently being pre-cleaned manually. In this case, it may also be recorded how long the endoscope has been in the pre-cleaning process. For example, if an endoscope is in a manual pre-cleaning station VR1 in reprocessing domain 150, the digital signal 532 being provided by the RFID readers in the examination room will encode an ID of the RFID tag on the endoscope and the ID of the RFID reader in manual pre-cleaning station VR1. Processor 510 can generate a timestamp indicating a receiving time of digital signal 530, and decode digital signal 532 to extract the encoded information. Processor 510 can run classification model 520 using the extracted information as input to determine a status that the endoscope is in undergoing manual pre-cleaning.

In one embodiment, processor 510 can store the outputs from classification model 520 in memory 512. Processor 510 can combine the information encoded in digital signals 530, 532 and the stored outputs into new training data that can be used for refining and retraining classification model 520.

Note that the classification being performed by having processor 510 execute classification model 520 is being performed autonomously, without human intervention. Thus, the autonomous classification can reduce the processing time and power when compared to systems that require human input. For example, the autonomous classification does not need to perform image processing to render the information encoded in digital signals 530, 532, on a display in order to wait for a user to manually classify the current status of the endoscopes.

After running the classification model 520 to determine the current status of the endoscopes, processor 510 can display at least some of the information encoded in digital signals 530, 532 and generate graphical components to be embedded in one or more presentation areas in user interface 105. For example, processor 510 can render the output from classification model 520 and render the information encoded in digital signals 530, 532 to generate a plurality of static and/or dynamic graphical components and embed the generated graphical components in user interface 105. Dynamic graphical components can be rendered by processor 510 using, for example, HTML5, CSS3 styling, SVG (Scalable Vector Graphics). Description of the graphical components will be provided in more detail below.

Processor 152 of each one of EDG1, EDG2 endoscope reprocessing devices can be configured to encode information about the type and progress of the selected reprocessing program, which also indicates the status of the endoscopes that are in the respective endoscope reprocessing device, in digital signal 550. Processor 152 can send digital signal 550 to processor 510, and processor 510 can run classification model 520 using the information encoded in digital signal 550 as input. In one embodiment, digital signal 550 can be among input telemetry being provided by reprocessing domain 150. A telemetry can be referred to as an automated, remote collection and wireless transmission of data from sensors, machinery, or software in reprocessing domain 150 to data processing system 102 for real-time monitoring and analysis. From the received data, the provisioning system 100 determines whether suitable endoscopes are available for each of the scheduled procedures. To do this, one or more compatible endoscope types with which the procedure can be performed are assigned to each planned procedure. At the same time, the number of endoscopes currently available of each endoscope type is determined. An endoscope is considered to be available if it has been completely reprocessed and has not exceeded its maximum permissible storage period in a storage cabinet. The availability of endoscopes can be among the information encoded in digital signal 550.

In addition to the endoscopes that are currently available, endoscopes that are currently in use or undergoing reprocessing but will be fully reprocessed and thus available by the time a procedure is scheduled to begin may also be taken into account in the availability check. Information indicating that a specific endoscope is currently in use or undergoing reprocessing, and their expected availability (e.g., expected completion time of reprocessing process) can be expected information being encoded by processor 152 in digital signal 550. Known durations of procedures and individual reprocessing processes are used here to make the most accurate prediction possible.

### Dashboard

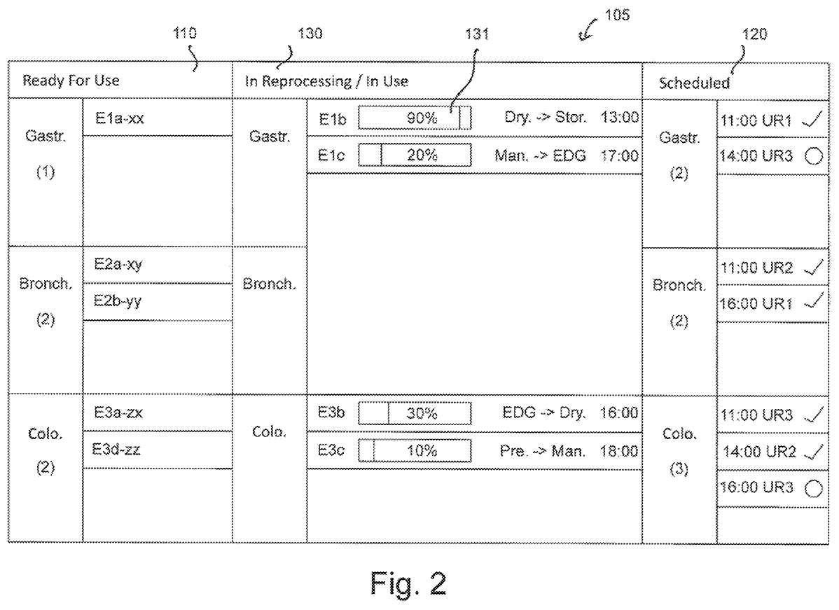

The provisioning system 100 further comprises a user interface 104 through which the determined information about the availability of endoscopes, that may be encoded in digital signal 550, is displayed. A key element of the user interface 104 is a graphical user interface 105 in which the information is intuitively displayed. This user interface 105 is also referred to as a “dashboard”. One possible embodiment of the user interface 105 is shown in FIG. 2.

The user interface 105 has a first presentation area 110 in which data about the endoscopes currently available for use is displayed. A second presentation area 120 displays data about scheduled procedures. A third presentation area 130 displays data about endoscopes currently undergoing reprocessing.

The first presentation area 110, the second presentation area 120, and the third presentation area 130 are arranged along a main direction, in the example shown along a horizontal line. Thereby, the third presentation area 130 can be arranged between the first presentation area and the third presentation area.

In the presentation areas 110, 120, 130, data relating to individual endoscopes and/or procedures are arranged along a second main direction, which can be perpendicular to the first main direction. In the example shown, the second main direction is a vertical line.

### First Presentation Area

To display the available endoscopes in the first presentation area 110, the type designations and/or serial numbers of the available endoscopes can be listed. The display can be sorted and/or grouped according to endoscope types. Additional grouping may be based on the type of procedure for which the respective endoscopes are suitable. For the groups thus formed, the number of endoscopes available in each group can be output to allow rapid acquisition of the relevant information by an observer.

The sorts and/or groupings to be applied can be configured by the user. Similarly, the user can define what information is to be displayed in the first presentation area. For example, the display of serial numbers of available endoscopes can be enabled or disabled. As further information, a remaining storage time for each available endoscope can be displayed.

In the example shown, the first presentation area 110 indicates that one gastroscope is available, namely gastroscope E1a with serial number E1a-xx. Furthermore, two bronchoscopes are available, namely bronchoscopes E2a, E2b with serial numbers E2a-xy and E2b-yy. In addition, two colonoscopes are E3a, E3d with serial numbers E3a-zy and E3d-zz.

### Second Presentation Area

The second presentation area 120 displays the scheduled procedures. The display can include, for example, the type of procedure, the planned start, and the examination room. Similar to the first presentation area, the second presentation area may be sorted and/or grouped, for example, by type of scheduled procedure. Again, a number of scheduled procedures may be displayed for each group of procedures for quick acquisition of information.

Also, for the second presentation area, a user can configure the type and amount of information displayed. For example, a user can use a filter to control the time period for which scheduled procedures are displayed and/or select only certain types of procedures for being displayed.

In the example shown, the second presentation area 120 indicates that two gastroscopies are scheduled, at 11:00 h in room UR1 and at 14:00 h in UR3. Furthermore, two bronchoscopies are scheduled, namely at 11:00 h in room UR2 and at 16:00 h in room UR1. Finally, three colonoscopies are scheduled, namely at 11:00 h in room UR3, at 14:00 h in room UR2, and at 16:00 h in room UR3.

### Third Presentation Area

The third presentation area 130 displays the endoscopes currently undergoing reprocessing. Here, for example, a remaining duration of the reprocessing process and/or the predicted time of availability can be displayed. In addition, the reprocessing step which the endoscope is currently undergoing can be displayed, as well as the remaining duration of the reprocessing step. Furthermore, a visualization 131 in the form of a percentage and/or a progress bar can be used to quickly and unambiguously detect the progress of the reprocessing process. Visualization 131 can include one or more dynamic graphical components rendered by data processing system 102 and embedded in the third presentation area 130 of user interface 105. The dynamic graphical components can be rendered by data processing system 102 using, for example, HTML5, CSS3 styling, SVG (Scalable Vector Graphics).

In addition to the endoscopes currently undergoing reprocessing, the third presentation area can also display endoscopes that are currently in use.

Similar to the first and second presentation areas, the endoscopes displayed in the third presentation area can be sorted and/or grouped by endoscope type and/or procedure type. Again, the number of endoscopes in each group can be displayed for quick reference.

In the example shown, gastroscopes E1b and E1c are currently undergoing reprocessing, with reprocessing of gastroscope E1 90% complete, so gastroscope E1b will be ready for use at 13:00. Gastroscope E1b is currently in the drying process and is awaiting transfer to storage. The reprocessing of gastroscope E1c is 20% complete and it will be ready for use at 17:00. Gastroscope E2c is currently in the manual pre-cleaning process and awaiting transfer to an endoscope reprocessor.

The display of information on the availability of endoscopes in the three presentation areas described above enables a user of the provisioning system to see at a single glance whether the required endoscopes are available or will be available on time for all procedures planned in the selected time period.

For example, it is immediately apparent that gastroscope E1a is ready for the gastroscopy planned at 11:00, and that gastroscope E1b, which is required for the gastroscopy planned at 14:00, will already be fully reprocessed at 13:00.

Bronchoscopes E2a and E2b are ready for use for the bronchoscopies scheduled at 11:00 and 16:00.

For the colonoscopies at 11:00 and 14:00, the E3a and E3d colonoscopes are ready for use. Furthermore, it is immediately apparent that colonoscope E3b will be ready for use at 16:00 and can be used in the colonoscopy scheduled for 16:00.

In order to support rapid visual recognition, the information in the respective presentation areas can be highlighted in color or provided with pictograms. For example, in the second presentation area 120, those scheduled procedures for which endoscopes are ready for use can be highlighted in green and/or provided with a “tick” symbol. Procedures for which the endoscopes are currently being reprocessed but are to be available in time can be highlighted in yellow and/or provided with a “circle” symbol. Procedures for which an endoscope is not expected to be available in time can be highlighted in red and/or marked with a “triangle” symbol.

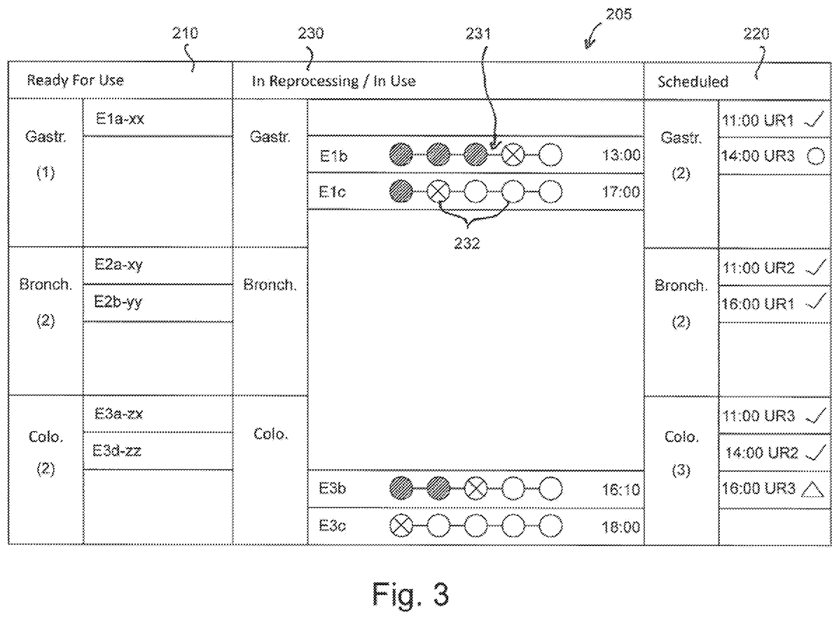

FIG. 3 illustrates another possible embodiment of the user interface 205. It again comprises a first presentation area 210, a second presentation area 220, and a third presentation area 230. Here, the first presentation area 210 and the second presentation area 220 are structured in the same way as the corresponding presentation areas 110, 120 of the user interface 105. The third presentation area 230, however, differs in layout from the third presentation area 130.

In the third presentation area 230, the reprocessing progress of the displayed endoscopes is represented by a visualization 231 in the form of a “string of pearls” with several visualization elements 232, in the example shown as nodes, each node representing a specific step of the reprocessing process. Here, the nodes are represented differently depending on whether the corresponding reprocessing step is still pending, currently being carried out, or already completed. For example, completed steps can be represented with a filled node and pending steps can be represented with a bordered node, while steps currently carried out can be represented with a cross, for example. Other representations are, of course, equally possible.

The graphical user interface can present suggested automated modifications and supporting evidence in dedicated presentation areas where an authorized operator can review and respond. Suggested parameter changes appear alongside the scheduled procedure listings in the second presentation area and include a short reasoning summary (for example: ‘Extend Drying: fused_wetness_score=0.82, dwell_time=180 s; recommended additional drying=300 s’). Operator options include Approve (automatically send command), Modify (edit numeric fields which updates the command preview), or Reject (log the rejection). If no action is taken within a defined operator_timeout (e.g., 30 s for non-critical recommendations, or a different preconfigured interval for safety-critical changes), the system follows the configured fallback behavior (e.g., do not apply the change and require manual re-scheduling, or apply a conservative automatic action). Presentation area color coding and pictograms described for quick visual status (green tick, yellow circle, red triangle) are used to highlight items that require attention, and each operator action is recorded in the control memory for traceability.

The graphical user interface may present recommended parameter adjustments and scope-allocation options in designated presentation areas. When the optimization engine produces a recommended modification, the GUI displays the recommendation together with an actionable interface element, such as a one-click button. When the operator selects the action, the system immediately generates the corresponding control command and transmits it to the reprocessing device or manual pre-cleaning station.

This integration allows rapid application of optimized parameter sets, reduces cognitive burden on staff, and minimizes opportunities for human error. The GUI may also show real-time confirmation messages, enabling users to verify that the reprocessing device accepted and applied the recommended changes.

In the third presentation area 230, the reprocessing progress of the displayed endoscopes is represented by a visualization 231 in the form of a “string of pearls” with several visualization elements 232, in the example shown as nodes, each node representing a specific step of the reprocessing process. Here, the nodes are represented differently depending on whether the corresponding reprocessing step is still pending, currently being carried out, or already completed. For example, completed steps can be represented with a filled node and pending steps can be represented with a bordered node, while steps currently carried out can be represented with a cross, for example. Other representations are, of course, equally possible.

Similar to the third presentation area 130 of FIG. 2, the third presentation area 230 of FIG. 3 also displays the time at which the corresponding endoscope will be ready for use.

As a further special feature, the display of the endoscopes undergoing reprocessing in the third presentation area 230 is shifted downwards to such an extent that it is displayed below the indication of the endoscopes ready for use in the first presentation area 210. This shift results in the endoscopes undergoing reprocessing in the third presentation area 230 being displayed at the same level as the planned procedures in the second presentation area 220 in which the respective endoscope is to be used, if the displays are sorted accordingly. The interrelated data in the presentation areas 210, 220, 230 are thus aligned in the second main direction, in the example in vertical direction. In this way, a user of the provisioning system 100 can even more easily see whether a suitable endoscope will be available in time for each planned procedure.

In the example shown in FIG. 3, the provisioning of the colonoscope E3b has been delayed by 10 minutes compared to the situation shown in FIG. 2. Such delays can occur, for example, when fluctuations in water pressure or water temperature of the water supply cause a dosing or heating process in an endoscope reprocessor to take longer than planned. Therefore, the colonoscopy scheduled for 16:00 is marked with a “triangle” symbol to indicate that an adjustment to the occupancy schedule may be necessary here.

Additional information can be used to increase the accuracy of the prediction.

### Duration and Progress of a Procedure

The average duration of certain standard procedures such as a gastroscopy or colonoscopy is fairly well known. When determining the expected duration of a procedure, additional historical data may be taken into account. For example, the durations of previous examinations of the same patient can be used, as well as examinations of other patients performed by the same physician and/or in the same examination room. This can significantly increase the prediction accuracy for the duration of the examination.

For this purpose, the durations of past procedures are stored in a database implemented in the data processing system 102. In this regard, personal data regarding the patient or physician may be suitably anonymized.

Now, in order to determine when a current procedure will be completed so that the endoscope used can be transferred to reprocessing, the provisioning system can apply various filters to the stored data, and then statistically evaluate the filtered data.

The main filters to be considered here are the type of procedure and the physician performing the procedure. An average value of the procedure duration can then be determined from the data filtered in this way. Alternatively, or additionally, a trend analysis can be performed, by which e.g., a learning curve of a physician is taken into account, during which the duration of the procedures slowly decreases.

However, deviations from the statistically determined duration may occur due to different anatomical conditions and/or complications. To detect such deviations early and determine their impact on endoscope availability planning, progress can be continuously determined during the procedure and transmitted to the provisioning system.

Standard endoscopic procedures are usually divided into predetermined sections, which are defined, for example, by reaching certain anatomical landmarks. Reaching such a landmark is usually documented by storing a still image of the anatomical landmark.

Based on the already stored still images, it is thus possible to estimate how far the procedure has progressed. Deviations from an average procedure progress can thus be detected at an early stage and can be taken into account in availability planning.

In some embodiments, still images captured during the endoscopic procedure include timestamps marking the final anatomical locations reached or the start of scope withdrawal. The system extracts this timestamp and uses it to estimate the end-of-procedure time. From this estimate, the system calculates a deadline by which the endoscope should arrive at a pre-cleaning station. If the scope has not been detected at any pre-cleaning location by the computed deadline, the provisioning system automatically generates a “START_PRECLEAN” instruction.

If the pre-cleaning station supports automated actions, the instruction may activate water-heating elements, initiate detergent-mixing motors, or prepare flushing cycles. For manual stations, the instruction may appear as a display prompt for the operator. All deadlines, triggers, and corresponding actions are stored in the control memory for auditability. By linking image timestamps to cleaning-workflow control, the system reduces delays and ensures adherence to recommended reprocessing intervals.

### Manual Pre-Cleaning

Before reprocessing by machine, endoscopes must be pre-cleaned manually to remove coarse contaminants. The duration of pre-cleaning is mainly dictated by predefined protocols, but can also vary depending on the specialist performing the pre-cleaning and/or the pre-cleaning station used.

As soon as an endoscope is registered at a pre-cleaning station, information is available to the provisioning system as to which specialist is responsible for pre-cleaning. The provisioning system may then use historical data to estimate the duration of the pre-cleaning process. For example, over a longer period of time, it can be determined how much time it took each specialist to pre-clean each type of endoscope. If necessary, more detailed estimates can also be made, which take into account a day of the week or a time of day. Likewise, only those pre-cleaning processes can be evaluated that were performed at the same pre-cleaning station.

Here, too, the determination is made by filtering and statistically evaluating stored data on past processes.

### Placement Within Endoscope Reprocessing Devices

Modern endoscope reprocessing devices are usually designed for reprocessing several endoscopes in one operation. Due to the fact that several endoscopes are reprocessed simultaneously, delays in the provision of individual endoscopes also affect other endoscopes which are to be reprocessed at the same time as the delayed endoscope.

Such transfer of delays can be detected by the provisioning system and taken into account in availability planning.

### Progress of Machine Reprocessing

The process duration of machine reprocessing is essentially determined by fixed reprocessing programs. Nevertheless, time deviations may occur.

For example, endoscope reprocessing devices, such as EDG1, EDG2, can perform a leakage test for the endoscopes to be reprocessed. For this purpose, the endoscopes are pressurized with a pressurized gas and the pressure curve is monitored. Processor 152, at each one of EDG1, EDG2, can be configured to set the pressure being used and to monitor the pressure curve. Processor 152 can set the pressure by setting a flow rate of a pump to build up the pressure to a predefined target pressure. Since the flow rate of the pump used to build up pressure can change over time, there may be gradual changes in the duration of the leakage test.

Other reasons for variations can be water pressure fluctuations or changes in water temperature that result in altered dosing or heating times. Such variations can be random or systematic. Similarly, gradual changes in the delivery or heating capacity of individual units of an endoscope reprocessing machine can be the cause of changes in process duration.

Processor 152 can be configured to log and monitor such variations and encode the variations in digital signal 550 such that the variations can be reported to processor 510 in FIG. 5.

In an aspect, endoscope reprocessing devices or machines can provide information about process progress via a data interface so that this information can be taken into account directly by the provisioning system 100. For example, processor 152 of EDG1, EDG2 can be configured to monitor and compile information of the reprocessing progress occurring at EDG1, EDG2. The communication of such progress information between processor 152 and processor 510 can allow processor 510 to obtain and analyze historical data using the machine learning models described herein to improve an accuracy of endoscope reprocessing.

For example, conventional endoscope reprocessing machines may not provide such progress information. Nevertheless, a fairly accurate estimate of process progress can be made here, for example, by evaluating historical data on how long a particular reprocessing program usually lasts on a particular endoscope reprocessing machine. Through this, systematic deviations from a usual process duration can also be predicted and taken into account by the provisioning system. The evaluation of historical data is similar to that described with respect to procedure duration or pre-cleaning.

### Finite-State Machine Architecture

In additional embodiments, the reprocessing machine may be described using a finite-state machine (FSM) in which each operational phase is represented as a discrete state with well-defined entry and exit conditions. Representative states include:
Idle: indicating no endoscope is present;
Pre-Cleaning: during which manual or automated preliminary cleaning occurs;
Automated Reprocessing: encompassing detergent wash, chemical disinfection, rinsing, and related actions;
Post-Rinse: representing transitional steps following chemical disinfection;
Drying: in which controlled airflow or heating removes residual moisture;
Ready or Storage: indicating that the endoscope meets all reprocessing requirements; and
Quarantine: used when anomalies or performance deviations require removal of the endoscope from circulation.

State transitions may be triggered by completion of a timed step, achievement of a sensor-verified threshold (e.g., temperature, concentration, pressure), or arrival of a machine-learning-generated control command. For example, a “SET_TIME” command containing a reduced exposure duration may cause the processor to shorten a wash or disinfection step, prompting the FSM to transition from the Automated Reprocessing state to the Post-Rinse state earlier than under default parameters. Likewise, an anomaly-driven “PAUSE_CYCLE” command may cause an immediate transition to the Quarantine state. The FSM structure enables predictable machine behavior, verifiable safety conditions, and a clear mapping between computed decisions and physical operations.

### Drying and Storage

After reprocessing is completed at EDG1, EDG2, endoscopes can be transferred to drying cabinets, such as TS1, TS2, TS3, for drying under controlled ambient conditions. The duration of the drying process is hardly subject to fluctuations. After drying, endoscopes are usually transferred to storage cabinets, where they are also stored under controlled environmental conditions until their next use. Here, the maximum permissible storage period is limited; after exceeding the storage period, an endoscope may have to be reprocessed.

### Measures in Case of Deviations

If the evaluation of the availability of the endoscopes provides indications that a suitable endoscope will not be available in time for a planned examination, measures may have to be taken to counteract this.

In a simple case like the situation shown in FIG. 3, where an endoscope will be delayed by a few minutes, the planned procedure can be postponed slightly. To do this, it may simply be necessary to inform the patient of the postponement.

However, in unfavorable cases, delaying a procedure can lead to a chain of further delays, which can cause resentment among patients and staff. Therefore, it may be helpful to provide other possible countermeasures against a delay. For this reason, the provisioning system 100 is configured to allow dynamic adjustment of reprocessing processes for endoscopes.

Typically, the steps to be performed during the reprocessing of an endoscope are fixed by protocols. These protocols specify, among other things, how long channels of an endoscope are to be cleaned with a brush during pre-cleaning, which exposure times, active agent concentrations, and process temperatures are to be observed during machine reprocessing, and under which ambient conditions an endoscope is to be dried. The appropriate protocols are usually specified by the endoscope manufacturer to ensure effective reprocessing.

The effectiveness of reprocessing is usually defined by the achieved reduction in the number of colony forming microorganisms (CFU, “Colony Forming Units”). In this regard, each step in a given reprocessing protocol results in a certain reduction in CFU, and the individual reductions add up or multiply to the total reduction achieved (the reduction is most often expressed logarithmically, e.g., in a number of powers of ten by which the number of CFU is reduced).

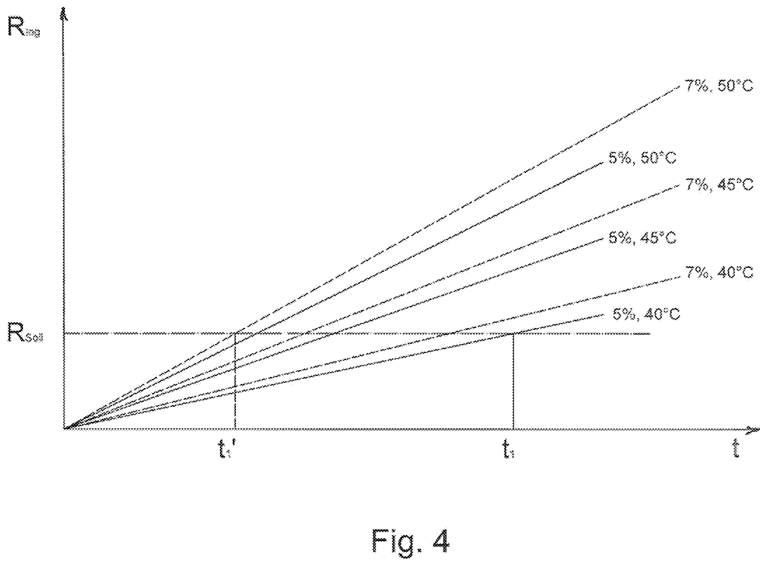

The provisioning system 100 includes a control memory 102a (or memory 102a) configured to store rules according to which the process parameters of the predetermined preparation steps can be changed without affecting the effectiveness of the preparation processes. In one embodiment, the control memory 102a may be part of the data processing system 102. In another embodiment, the control memory can be outside of data processing system 102, such as being located in a server configured to communicate remotely and wirelessly with data processing system 102. For example, the control memory 102a may store a relationship between individual process parameters of an individual reprocessing step and its effectiveness in the form of a performance curve field. Such a performance curve field is exemplarily shown in FIG. 4.

FIG. 4 shows the relationship between a reduction in CFU in a disinfection step in an endoscope reprocessing machine. The reduction R is plotted logarithmically as R_{log} on the vertical axis of the characteristic curve. The process time t is plotted linearly on the longitudinal axis.

The provisioning system can include an optimization engine that computes modified process parameters based on declared performance curves and constraints. In one embodiment the optimization problem is stated as: minimize total_cycle_time subject to achieving a minimum microbial reduction M_req as read from the performance-curve field, actuator capability limits, and material safety constraints (e.g., temperature≤Tmax). Decision variables may include treatment_time_seconds and treatment_temperature_degc. A representative solver approach is an integer or mixed-integer program (for example, discretize treatment_time into 10 s steps and treatment_temperature into 1° C. steps and solve with integer linear programming) or a bounded exhaustive search for small domains. As a worked example, given a performance curve that indicates 2 log reduction at 50° C. in 300 s and 3 log reduction at 55° C. in 200 s, the optimizer may select 55° C. and 200 s to meet a 3-log requirement while minimizing cycle time. Herein, reduction of the required time from 300 s to 200 s may help reducing or eliminating any delay in endoscope availability, whereas increase of the CFU reduction may help compensate possible shortcomings of preceding or following reprocessing steps. A translator module being run by processor 510 then makes deterministic actuator mappings to set heater setpoint=55° C. and timer=200 s and issues the commands for acknowledgment and audit. The optimization engine can be implemented by having processor 510 run an optimization model 524, described below.

By way of example, in the disinfection step, the endoscope is exposed to a disinfection solution of a given concentration for the process time t at a given temperature. The performance curves in FIG. 4 show the course of the reduction of the CFU at different concentrations and temperatures. Solid lines show the time dependent reduction of CFU at a concentration of the disinfectant solution of 5%, and at temperatures of 40° C., 45° C., and 50° C. Dashed lines show the time dependent reduction at a concentration of 7%, also at temperatures of 40° C., 45° C., and 50° C. It can be seen that a given reduction of Rₛₒₗₗ is achieved after a regular process time of t1, when a concentration of 5% and a temperature of 40° C. are set. In contrast, the same reduction Rₛₒₗₗ is achieved after a much shorter process time t1′ if a concentration of 7% and a temperature of 50° C. are set.

The performance curve field shown in FIG. 4 serves only as an example. Similar performance curve field can be drawn up for other preparation steps.

In the event of a delay in the provision of a required endoscope, as shown in FIG. 3 for endoscope E3b, the provisioning system 100 may be arranged to propose measures to compensate for or reduce the effects of the delay.

For this purpose, the provision system can execute a computer program which examines the performance curves stored in the control memory 102a to determine whether a reprocessing step still to be performed for the endoscope concerned can be modified in such a way that the delay is compensated. Safety limits of individual parameters can be observed in order to avoid damage to the endoscope.

Identified options for adjusting the reprocessing process may be offered to a user of the provisioning system for selection. The user can then decide, e.g., based on further considerations, whether or not to perform a modification. In doing so, the user can weigh economic effects of the modification, such as increased wear and tear on the endoscope, against the effects of the delay, such as disruption of scheduled procedures.

In addition to separate modification of individual reprocessing steps in which the reduction in CFU is maintained for each reprocessing step, successive reprocessing steps can also be modified such that a change in reduction at one step is offset by an opposite change in reduction at another step.

The determination and offering of adjustments can be triggered by the user by activating a button in the graphical user interface. For this purpose, for example, a data element in the second presentation area 120, 220 representing the procedure affected by a delay may be implemented as an interactive button, the activation of which by a graphical input device such as a mouse, or by touch when the user interface is displayed on a touch-sensitive screen, triggers the determination. Determined adjustments can then be displayed in the form of a list, e.g., in a “pop-up” window. The entries of this list may in turn be implemented as interactive buttons, upon activation of which the corresponding customization is implemented.

To implement an adjustment, the provisioning system 100 may send one or more control commands to an affected endoscope reprocessing device, and/or transmit instructions in text form to a manual reprocessing station for display.

As a representative example, consider endoscope S123 placed into reprocessor R1 at time t₀. At t₀+8 minutes, telemetry indicates that the device is in the wash phase. A machine-learning model predicts a ready-for-use time of T_ready=t₀+21 minutes with 85% confidence. A procedure P45 is scheduled to begin at t₀+30 minutes. To ensure on time availability, an optimization module evaluates performance curve fields and determines that decreasing the final rinse duration by 2 minutes and increasing the drying temperature by 5° C. will still achieve the required hygiene threshold.

The system writes the updated parameters into the control memory and generates a “SET_PARAMS” command that includes these modifications. The reprocessing device acknowledges command receipt and applies the new settings. The provisioning system records the acknowledgment, updates the FSM, and monitors subsequent telemetry until the cycle completes. The full decision history including inputs, predictions, optimizer outputs, parameter changes, commands, and acknowledgments is preserved in the audit trail, ensuring transparency and verifiability.

Referring to FIG. 5, in another embodiment, processor 510 can be configured to use the timestamps indicating receiving times of digital signals 530, 532 to detect anomalies associated with the current status of the endoscopes and to predict future events of the endoscopes. As shown in FIG. 5, memory 512 can be further configured to store a file 516 that is a digital representation of an anomaly detection model 522. In one embodiment, anomaly detection model 522 can be an auto-encoder, or a combination of statistical control-charting and drift detector, that can detect whether the output of classification model 520 deviates from an expected outcome (e.g., performance curve in FIG. 4 and stored in memory 102a). File 516 can be, for example, a binary serialized file that includes a structured collection of data representing parameters in anomaly detection model 522. File 516 can also include program code, that are machine readable codes not interpretable by humans, that define the decision boundaries and comparison criteria for detecting deviations between outputs from classification model 520 and expected outcomes.

Processor 510 can generate time series data of each endoscope based on the timestamps of receiving digital data 530, 532. The time series data of an endoscope can indicate the times, including start times, end times, and durations of various status such as in-use, manual pre-cleaning process, reprocessing (including a disinfection step), drying, and storage. Processor 510 can input the generated time series data in anomaly detection model 522, and run anomaly detection model 522 to compare the time series data with an expected outcome.

In one embodiment, processor 510 can be further configured to run a progress estimation model 542. Progress estimation model 542 can be a convolution neural network (CNN) implemented with a regression model. Progress estimation model 542 can take stored or live videos and/or images (including frames of videos), time series data (historical and current) as input, and infer an estimated progress indicating the current procedure or reprocessing step, remaining or future steps, progress of completion (e.g., completion percentage) and the estimated durations for the indicated steps. The historical time series data can be historical progress curves per procedure type, or physician, or examination room. In one embodiment, a file 540 stored in memory 512 representing progress estimation model 542 can be a binary serialized file that includes a structured collection of data representing parameters in progress estimation model 542. File 540 can include program code, that are machine readable codes not interpretable by humans, that define the parameters such as the CNN weighs, layers and their connections, size of the CNN layers, inputs and outputs, etc.

Processor 510 run the progress estimation model 542 to generate prediction of timings of current and future events for the endoscopes. For example, if an endoscope is undergoing a drying step currently, and the historical time series data indicates an expected drying duration for the endoscope, then processor 510 can run progress estimation model 542 that outputs an estimated remaining time to complete the drying, and also estimate duration of any remaining steps. If the estimated remaining time to complete the drying does not exceed the end of the expected duration of the drying step, then processor 510 can determine that no changes are needed to the current scheduling of reprocessing procedure. If the estimated remaining time to complete the drying exceeds the end of the expected duration of the drying step, then processor 510 can send one or more digital signals 554 encoding control commands for processor 152 to modify the current scheduling of reprocessing procedure, such as by modifying the settings of the reprocessing process in reprocessing domain 150. The control commands being encoded in the digital signals 554 can be commands specific to the reprocessing devices, such as mechanical set points for setting environmental parameters being produced by the reprocessing devices such as set points for temperatures and pressure. In one embodiment, processor 510 can determine the modification to the settings by running an optimization model (described below). The utilization of the progress estimation model 542 can allow deviations from expected outcomes to be detected early for processor 510 to update expected endoscope handoff times, reducing allocation conflicts and improving ready-time accuracy.

In one embodiment, the estimated progress from progress estimation model 542 can be expressed as a confidence score or probability. For example, progress estimation model 542 can output a first estimation of progress that has a 95% likelihood to be accurate and a second estimation of progress that has an 85% likelihood to be accurate. Both outputs can be stored in memory 512 and can be reused for retraining progress estimation model 542. For example, if an endoscope's reprocessing procedure times align with the second estimation indicating 85% accuracy, then processor 510 can generate training data indicating that there is a new instance where the second estimation from progress estimation model 542 is accurate over the first estimation.

In one embodiment, progress estimation model 542 can receive still images or videos to estimate a progress in the work cycle of an endoscope. For example, progress estimation model 542 can be trained using videos that are labeled with progress of a procedure in the examination room. Progress estimation model 542 can receive live videos captured by the endoscope during an examination and infer remaining time to complete the ongoing procedure and estimate a start time of the manual pre-cleaning procedure and subsequent steps.

In another example, if the time series data based on the current state of an endoscope shows an endoscope has been in the disinfection step for a time period T1, but the expected outcome indicates an expected disinfection duration for the endoscope shall be a time period T2 that is less than T1, then anomaly detection model 522 can generate an output indicating an anomaly.

In a further embodiment, the provisioning system includes an optimization module designed to compute improved reprocessing parameters that satisfy operational, hygienic, and workflow constraints. The module defines an objective function such as minimizing total cycle duration, minimizing lateness relative to scheduled procedures, or maximizing throughput under resource limitations. Decision variables may include exposure times, flow-rate settings, chemical-agent concentrations, drying-temperature levels, and logical allocation variables specifying which reprocessing device handles which endoscope.

Constraints applied during optimization may include minimum microbial-reduction thresholds derived from performance curve fields; maximum allowable temperatures and concentrations based on endoscope material compatibility; capacity limits of the reprocessing device; and clinical scheduling requirements. The optimization engine may be implemented using integer linear programming, mixed-integer programming, or reinforcement-learning-assisted heuristics. Following computation of the optimal parameter set, the system writes the selected parameters to the control memory and initiates generation of corresponding control commands. This ensures that every recommended parameter change is both effective and compliant with hygiene standards.

In one embodiment, processor 510 can use the detected anomaly from anomaly detection model 522 and/or the estimated progress from progress estimation model 542 to generate commands for processor 152 to modify an ongoing or pending reprocessing processes by which a delay in provisioning the endoscope can be reduced. For example, processor 510 can be configured to input the anomaly detected from running anomaly detection model 522 into an optimization model 524. Optimization model 524 can be a constraint solver (e.g., integer linear programming (ILP)) solvers or machine learning-assisted heuristic that outputs assignments and suggested swap paths (e.g., recommendation to modify the reprocessing). As shown in FIG. 5, memory 512 can be further configured to store a file 518 that is a digital representation of optimization model 524. In one embodiment, optimization model 524, when being run by processor 510, can generate and output data indicating at least one recommendation. Processor 510 can apply the outputted data to generate updated process parameters for reprocessing steps (e.g., temperature, concentration, time, which may affect hygiene and material compatibility) that are already being implemented to meet throughput or availability targets while respecting effectiveness constraints. Processor 520 can generate commands indicating the updated process parameters, and encode the commands in a digital signal 550. Processor 520 can send digital signal 550 to processor 152 in reprocessing domain 150. For example, referring to FIGS. 4 and 5, a current settings of the disinfection step in the reprocessing is a concentration of 5% and a temperature of 40° C. and the disinfection duration T1 is greater than t1′. Thus, anomaly detection model 522 will output an anomaly. Processor 510 can input data representing the detected anomaly into optimization model 526 and run optimization model 526. Optimization model 526 can output a recommendation to change the settings to achieve a sooner completion of the disinfection step while achieving given reduction of Rₛₒₗₗ, such as modifying the settings to a concentration of 7% and a temperature of 50° C. in order to achieve a given reduction of Rₛₒₗₗ sooner than time t1 shown in FIG. 4. Processor 510 can encode the updated parameters, such as updated concentration of 7% and updated temperature of 50° C., in digital signal 550 and send digital signal 550 to processor 152.

In another embodiment, a leakage test being performed by one or more of EDG1, EDG2, can be monitored by processor 510. If the detected anomaly from anomaly detection model 522 and/or the estimated progress from progress estimation model 542 shows an ongoing leakage test is taking longer than an expected duration, processor 510 can generate a command and encode the command as a digital signal that can be transmitted to processor 152 of the reprocessing device performing the leakage test. The command being encoded in the digital signal can command processor 152 to change a flow rate of a pump to adjust the pressure being used in the leakage test to speed up the leakage test. The collaboration between processor 152 and processor 510 can address random and unexpected change in pressure being used in leakage tests.

In another embodiment, in addition to the performance curve shown in FIG. 4, optimization model 524 can be trained by various other types of data such that in the inference phase, and optimization model 524 can use these various types of data to generate recommendations. For example, control memory 102a can be further configured to store current and historically known backlogs (and solutions to the historically known backlogs), scope material and/or conditions, predefined effectiveness thresholds. In one embodiment, the data being stored in control memory 102a for training, or to be used by, optimization model 524 can be stored as one or more look-up tables that map settings such as time, concentration, temperature to hygiene and material compatibility. The usage of look-up tables can allow processor 510 to run optimization model 524 with reduced computational load, such as avoiding the need to perform complex, or nonlinear calculations, thus preserving power consumption. Processor 510 can be configured to generate a query that is designated for specific look-up tables in control memory 102a. For example, processor 510 can generate a query, that has a key value pair including a primary key and an associated value, that can be used for searching for an output in a specific look-up table that includes a predefined category of the primary key.

In brief, classification model 520 can determine the current state of endoscopes. Anomaly detection model 522 can determine deviations between current state and expected outcomes. Optimization model 524 can optimize the settings of the reprocessing based on anomalies. Progress estimation model 542 can estimate remaining progress and procedures in the reprocessing. By using an ordered combination and of different machine learning models, data processing system 102 can provision work cycle of endoscopes and based on the provisioning, provide automatic adjustments to optimize the work cycle between usage, cleaning and reprocessing. The automatic adjustments can provide automatic modification to process parameters for reprocessing steps (e.g., temperature, concentration, time; which may affect hygiene and material compatibility) to meet throughput or availability targets while respecting effectiveness constraints, without human intervention, hence improving the speed to make adjustments when necessary. Further, the utilization of machine learning models can reduce human manual error, providing improvement in accuracy of endoscope work cycle provisioning systems. The automatic modifications provide concrete physical control outputs (parameter set and control commands) that change how reprocessing machines or manual stations operate, improving throughput while monitoring effectiveness, which yields a measurable transformation of machine behavior and physical process outcomes. Still further, by using processor 510 to run machine learning models on instantaneous data from processor 152 of the reprocessing devices EDG1, EDG2, processor 510 can analyse the instantaneous data and provide commands to control the reprocessing devices to improve an efficiency of the reprocessing and provisioning of endoscopes. The use of processor 510 to analyze and provide commands to improve the process can provide added functionalities to the reprocessing with minimal modifications to hardware in processors of existing reprocessing devices.

In one embodiment, when a work cycle, starting from a start of a procedure in an examination room to an ending where an endoscope goes into storage, is completed, the outputs from classification model 520, anomaly detection model 522, optimization model 524, and progress estimation model 542 can be stored in memory 512 and/or memory 102a. The outputs can be used for adjusting the expected outcomes, such as performance curves and look-up tables, stored in memory 102a. The outputs can also be used for retraining the classification model 520, anomaly detection model 522, optimization model 524, and progress estimation model 542 to improve an accuracy of these models.

Further, to ensure fully traceable operation, processor 510 may create a structured decision record in the control memory 102a for machine learning models stored in memory 512 that performs machine learning inference, optimization output, or operator initiated action. The structured decision records stored in control memory 102a may include, but not limited to:
a timestamp;
a unique decision identifier;
the full list of telemetry inputs used for inference;
still image or progress indicator identifiers;
the model version;
a confidence score;
a feature importance vector or indication of the most influential inputs;
the selected process parameter set;
the generated control command;
the device's acknowledgment; and
any operator follow up actions.

These records may be presented in a GUI, such a user interface 105, as log entries, tables, or expandable panels, giving authorized personnel the ability to review the reasoning behind every parameter modification. The structured storage of control artifacts supports safety certification, debugging, training of updated models, and quality assurance processes.

In an implemented embodiment the control memory stores structured decision records with a defined schema. Typical fields include: decision_id (unique), timestamp_utc, input_summary (e.g., fused_state_score and key sensor feature values), model_or_optimizer_version, suggested_parameters (named fields such as flow_l_per_min, temperature_degc, duration_seconds), command_payload (formatted message sent to device), device_acknowledgment (status code, ack_timestamp), operator_action (approved/modified/overridden and operator_id), and audit_hash (cryptographic digest of the record). Records are indexed for efficient retrieval by endoscope serial number, process batch, and date/time, and presented in the GUI as logs, tables, or expandable panels for authorized reviewers.

Processor 510 may implement a closed loop adaptive control process that continuously evaluates telemetry from the reprocessing devices (EDG1, EDG2) and dynamically adjusts machine parameters. In one representative sequence, processors 152 of the reprocessing device transmits telemetry, encoded in digital signals 550, including pump pressures, water temperatures, disinfectant concentrations, valve position indicators, and time elapsed values. Upon receiving this data, the processor forwards the telemetry to a trained machine learning model. The model outputs updated process parameters such as modified rinse duration, temperature setpoints, or flow rate adjustments based on inference from the incoming sensor stream.

The updated parameters are logged in the control memory 102a, together with the input telemetry encoded in digital signal 550 and inference metadata. Processor 510 can generate a control command embedding the new parameters, encode the control command in digital signals 554, and transmit it to processor 152 of the reprocessing device. Processor 152 can control the reprocessing device to execute the command by adjusting its actuators accordingly and return a confirmation message to processor 510. The confirmation is recorded by processor 510 in memory 102a, and a finite state machine (FSM) state is updated, and the new telemetry is used in the next inference cycle, such as being used by the machine learning models in memory 512. This adaptive pipeline allows the reprocessing device to respond in real time to fluctuating environmental conditions, device performance variations, or workflow constraints.

In one or more embodiments, when processor 510 determines that a modification to one or more reprocessing parameters is appropriate, processor 510 automatically generates a structured control command conforming to the actuator interface protocol used by the endoscope reprocessing devices EDG1, EDG2. Each control command includes a command identifier, such as “SET_TEMPERATURE”, “SET_EXPOSURE_TIME”, “SET_CONCENTRATION”, “SET_FLOW_RATE”, or “SET_DRYING_DURATION”, and an associated parameter set containing one or more updated values that are to be applied by the reprocessing machine. Processor 510 can encode these structured control commands in digital signals 554.

Before transmission, processor 510 can write the updated parameter set into a designated field of the control memory (e.g., memory 102a), together with a timestamp, a model version identifier, and a decision context record linking the modification to the telemetry or operational conditions that triggered the adjustment. Processor 510 then encodes (e.g., in digital signals 544) the command in a structured format required by the communication interface of the reprocessing devices EDG1, EDG2. The structured format may include a header identifying the destination device, a command opcode, a serialized list of updated parameters, and an error checking sequence such as a cyclic redundancy check (CRC). After encoding these information in digital signals 554, processor 510 can transmit digital signals 554 to processors 152 of reprocessing devices EDG1, EDG2.

In one example implementation, processor 510 can implement a translator module that converts optimizer outputs (output from optimization model 524) and fuse state information into explicit actuator parameters and a machine readable command message. For example, optimizer targets such as target_flow_l_per_min (e.g., a target flow rate) and target_temperature_degc (e.g., a target temperature setting) are mapped deterministically into actuator parameters for EDG1, EDG1, including VALVE_OPEN_MS (integer milliseconds), PUMP_PWM_PERCENT (0-100), and HEATER_SETPOINT_DEGC (degrees Celsius) by simple scaling functions (for example VALVE_OPEN_MS=round(base_valve_ms×target_flow_l_per_min/nominal_flow_l_per_min)). Commands are formatted as JSON objects with fields {cmd_id, device_id, actuator_type, parameters, timestamp_utc, required_ack_deadline_ms, checksum}. When processor 152 of a reprocessing device (e.g., EDG1, EDg2) receives digital signals 554 encoding the actuator parameters, processor 152 can return an acknowledgment token within a target duration, which can be set by a parameter required_ack_deadline_ms that can also be encoded in digital signals 554. If processor 510 does not receive the acknowledgment from processor 512, processor 510 can run the translator module to retry the conversion according to a configured retry policy. If a number of failures to receive the acknowledgement reaches a predefined number, then processor 510 can run the translator module to place the associated decision record into SAFE-HOLD for operator or user review. Processor 510 can further store the original command, any retries, and the device acknowledgment together in control memory 102a to create a verifiable link from input telemetry and optimizer decisions to enacted actuator changes.

When processor 152 returned an acknowledgment signal indicating successful receipt or identifying an error condition, processor 510 can store the acknowledgment in the control memory 102a alongside the issued command, completing a full audit trail that links inputs, decisions, commands, and device confirmations. This structured exchange ensures deterministic execution of changes, supports safe machine operation, and provides verifiable traceability for regulatory compliance.

Further, Referring to FIGS. 2 and 3, processor 510 can selectively display the outputs from the machine learning models, and/or display static or dynamic graphical components generated based on the model outputs, in user interface 105. For example, the IDs of the endoscopes being classified by classification model 520 as “ready for use” can be listed in first presentation area 110 and current reprocessing status of endoscopes, such as drying or storage, can be displayed in second presentation area 120. Processor 510 can use the anomalies detected by anomaly detection model 522, the recommendation from optimization model 524 and the estimate progress from progress estimation model 542 to modify the schedule being displayed in third presentation area 130. Also, processor 510 can display the progress of current status, outputted by progress estimation model 542, as dynamic graphical components such as progress bars (showing percentage) in second presentation area 120. In one embodiment, the progress being shown in second presentation area 120 and/or schedule being shown in third presentation area 130 can serve as inputs to anomaly detection model 522, optimization model 524 and/or progress estimation model 542 as inputs in order to determine necessary modifications to the schedule.

The systems and methods described herein can adapt to a situation where an endoscope will not be available in time for a scheduled procedure. In such a situation, the system can determine one or more modifications to ongoing or pending reprocessing processes by which a delay in provisioning the endoscope can be reduced. Modification(s) to ongoing or pending reprocessing processes by which a delay in provisioning the endoscope can be reduced increases the likelihood that the endoscope can be available for use for a scheduled procedure.

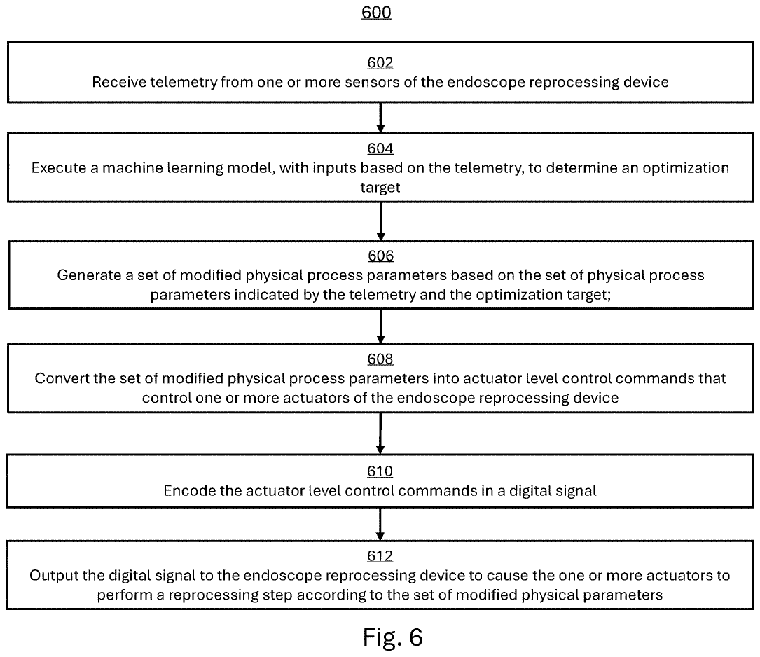

FIG. 6 illustrates a process for adaptively operating an endoscope reprocessing device in one embodiment. Process 600 can include one or more operations, actions, or functions as illustrated by one or more of blocks 602, 604, 606, 608, 610, and 612. Although illustrated as discrete blocks, various blocks may be divided into additional blocks, combined into fewer blocks, eliminated, performed in a different order, or performed in parallel, depending on the desired implementation.

Process 600 can be performed by one or more processors comprising hardware, such as processor 510 described in the present disclosure, for adaptively operating an endoscope reprocessing device.

Process 600 can begin at block 602. At block 602, a processor can receive telemetry from one or more sensors of the endoscope reprocessing device. The telemetry can indicate a set of physical process parameters currently being used in an operation of the endoscope reprocessing device.

Process 600 can proceed from block 602 to block 604. At block 604, the processor can execute a machine learning model, with inputs based on the telemetry, to determine an optimization target that indicates target physical process parameters for optimizing the operation of the endoscope reprocessing device.

Process 600 can proceed from block 604 to block 606. At block 606, the processor can generate a set of modified physical process parameters based on the set of physical process parameters indicated by the telemetry and the optimization target.

Process 600 can proceed from block 606 to block 608. At block 608, the processor can convert the set of modified physical process parameters into actuator level control commands that control one or more actuators of the endoscope reprocessing device. In one embodiment, the one or more actuators can include at least one pump motor, valve, heater element, or chemical dosing mechanism.

Process 600 can proceed from block 608 to block 610. At block 610, the processor can encode the actuator level control commands in a digital signal; and

Process 600 can proceed from block 610 to block 612. At block 612, the processor can output the digital signal to the endoscope reprocessing device to cause the one or more actuators to perform a reprocessing step according to the set of modified physical parameters.

In one embodiment, process 600 can further include adjusting valve timing in response to predicted flow rate changes.

In one embodiment, process 600 can further include running the at least one machine learning model to detect anomalies in pressure curves during leak testing and issuing a specific actuator level control command indicating to pause a reprocessing cycle in response to detection of the anomalies in pressure curves during leak testing.

In one embodiment, process 600 can further include storing the set of modified physical parameters and a command acknowledgment in a control memory.

In one embodiment, process 600 can further include using a structured control artifact record comprising timestamp, inputs, model version, confidence score, selected parameter set, and issued commands.

In one embodiment, process 600 can further include running a machine learning model that is an optimization model with an objective function, decision variables, and constraints derived from performance curve fields.

In one embodiment, process 600 can further include performing sensor fusion on humidity and optical signals, among the telemetry, to determine a wetness condition and issuing a specific actuator level control command indicating an extension of drying when the wetness condition indicates residual wetness.

In one embodiment, a non-transitory computer readable medium can store instructions that cause a processor to perform process 600. A computer program product embodiment disclosed herein is a term used for describing any set of one or more non-transitory computer-readable storage medium collectively included in a set of one or more storage devices that collectively include machine readable code corresponding to instructions and/or data for performing computer operations specified in the computer program products. A storage device is a tangible device that can retain and store instructions for use by a computer processor. A computer readable storage medium may be an electronic storage medium, a magnetic storage medium, an optical storage medium, an electromagnetic storage medium, a semiconductor storage medium, a mechanical storage medium, or any suitable combination of the foregoing. A computer readable storage medium, as disclosed herein, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media.

While there has been shown and described what is considered to be preferred embodiments of the invention, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

What is claimed is:

1. A system for operating an endoscope reprocessing device, comprising:
a processor comprising hardware; and
a memory storing instructions and at least one machine learning model;
wherein the processor is configured to:
receive telemetry from one or more sensors of the endoscope reprocessing device, wherein the telemetry indicates a set of physical process parameters currently being used in an operation of the endoscope reprocessing device;
execute the at least one machine learning model, with inputs based on the telemetry, to determine an optimization target that indicates target physical process parameters for optimizing the operation of the endoscope reprocessing device;
generate a set of modified physical process parameters based on the set of physical process parameters indicated by the telemetry and the optimization target;
convert the set of modified physical process parameters into actuator level control commands that control one or more actuators of the endoscope reprocessing device;
encode the actuator level control commands in a digital signal; and
transmit the digital signal to the endoscope reprocessing device to cause the one or more actuators to perform a reprocessing step according to the set of modified physical parameters.

2. The system of claim 1, wherein the one or more actuators comprise at least one pump motor, valve, heater element, or chemical dosing mechanism.

3. The system of claim 1, wherein the processor is configured to adjust valve timing in response to predicted flow rate changes.

4. The system of claim 1, wherein the processor is configured to run the at least one machine learning model to detect anomalies in pressure curves during leak testing.

5. The system of claim 4, wherein the processor is configured to issue a specific actuator level control command indicating to pause a reprocessing cycle in response to detection of anomalies in pressure curves during leak testing.

6. The system of claim 1, wherein the processor is configured to store the set of modified physical parameters and a command acknowledgment in a control memory.

7. The system of claim 1, wherein the processor is configured to use a structured control artifact record comprising timestamp, inputs, model version, confidence score, selected parameter set, and issued commands.

8. The system of claim 1, wherein the processor is configured to run a machine learning model that is an optimization model with an objective function, decision variables, and constraints derived from performance curve fields.

9. The system of claim 1, wherein the processor is configured to perform sensor fusion on humidity and optical signals, among the telemetry, to determine a wetness condition.

10. The system of claim 9, wherein the processor is configured to issue a specific actuator level control command indicating an extension of drying when the wetness condition indicates residual wetness.

11. A method for adaptively operating an endoscope reprocessing device, comprising:
receiving telemetry from one or more sensors of the endoscope reprocessing device, wherein the telemetry indicates a set of physical process parameters currently being used in an operation of the endoscope reprocessing device;
executing a machine learning model, with inputs based on the telemetry, to determine an optimization target that indicates target physical process parameters for optimizing the operation of the endoscope reprocessing device;
generating a set of modified physical process parameters based on the set of physical process parameters indicated by the telemetry and the optimization target;
converting the set of modified physical process parameters into actuator level control commands that control one or more actuators of the endoscope reprocessing device;
encoding the actuator level control commands in a digital signal; and
outputting the digital signal to the endoscope reprocessing device to cause the one or more actuators to perform a reprocessing step according to the set of modified physical parameters.

12. The method of claim 11, wherein the one or more actuators comprise at least one pump motor, valve, heater element, or chemical dosing mechanism.

13. The method of claim 11, further comprising adjusting valve timing in response to predicted flow rate changes.

14. The method of claim 11, further comprising:
running the at least one machine learning model to detect anomalies in pressure curves during leak testing; and
issuing a specific actuator level control command indicating to pause a reprocessing cycle in response to detection of the anomalies in pressure curves during leak testing.

15. The method of claim 11, further comprising storing the set of modified physical parameters and a command acknowledgment in a control memory.

16. The method of claim 11, further comprising using a structured control artifact record comprising timestamp, inputs, model version, confidence score, selected parameter set, and issued commands.

17. The method of claim 11, further comprising running a machine learning model that is an optimization model with an objective function, decision variables, and constraints derived from performance curve fields.

18. The method of claim 11, further comprising:
performing sensor fusion on humidity and optical signals, among the telemetry, to determine a wetness condition; and
issuing a specific actuator level control command indicating an extension of drying when the wetness condition indicates residual wetness.

19. A non-transitory computer readable medium storing instructions that cause a processor to perform the method of claim 11.

20. A system comprising:
an endoscope reprocessing device comprising:
one or more actuators configured to perform at least one reprocessing step, in an operation of the endoscope reprocessing device, according to actuator level control commands;
one or more sensors configured to generate telemetry indicating a set of physical process parameters currently being used in the operation of the endoscope reprocessing device;
a processor comprising hardware, the processor being connected to the endoscope reprocessing device through a network; and
a memory storing instructions and at least one machine learning model;
wherein the processor is configured to:
receive the telemetry from the one or more sensors, via the network, of the endoscope reprocessing device;
execute the at least one machine learning model, with inputs based on the telemetry, to determine an optimization target that indicates target physical process parameters for optimizing the operation of the endoscope reprocessing device;
generate a set of modified physical process parameters based on the set of physical process parameters indicated by the telemetry and the optimization target;
convert the set of modified physical process parameters into a set of actuator level control commands that control the one or more actuators of the endoscope reprocessing device;
encode the set of actuator level control commands in a digital signal; and
transmit the digital signal to the endoscope reprocessing device; and
the one or more actuators of the endoscope reprocessing device being further configured to perform the at least one reprocessing step according to the set of actuator level control commands encoded in the digital signal.
